(19) United States
(12) Patent Application Publication
(10) Pub. No.: US 2024/0070818 A1
(43) Pub. Date: Feb. 29, 2024

(54) Image synchronizing method, system and electronic equipment

(71) Applicant: ZHUHAI DI PU MEDICAL TECHNOLOGY CO., LTD., Zhuhai (CN)

(72) Inventors: Chongwei CHI, Zhuhai (CN); Kunshan HE, Zhuhai (CN)

(21) Appl. No.: 18/071,061

(22) Filed: Nov. 29, 2022

(30) Foreign Application Priority Data
Aug. 31, 2022 (CN) 202211053082.7

(51) Int. Cl. G06T 5/50; G06T 1/00; G06T 5/00; G06T 7/33

(52) U.S. Cl. CPC G06T 5/50; G06T 1/0007; G06T 5/003; G06T 7/33; G06T 2207/10064; G06T 2207/20192; G06T 2207/20221; G06T 2207/30096

(57) ABSTRACT

The present disclosure provides an image synchronizing method, a system and a piece of electronic equipment, which can invoke a preset frame synchronization parameter when receiving fluorescence image data; and white light image data corresponding to one memory chip is invoked from N memory chips based on the frame synchronization parameter, and the white light image data is fused with the fluorescence image data, so as to obtain a first fusion image. Under a case that the white light image and the fluorescence image generate difference, this method may compensate for the difference between the white light image and the fluorescence image in a manner that the white light image data after the difference is invoked through the frame synchronization parameter, thereby ensuring an imaging effect of a fluorescence imaging system.

### TECHNICAL FIELD

The present disclosure relates to the field of electronic technologies, and in particular to an image synchronizing method, a system and a piece of electronic equipment.

### BACKGROUND

A fluorescence endoscope system is an important device for identifying and positioning a human lesion tissue, and the accurate resection for the lesion tissue may be implemented by virtue of this device. When using this device, in order to accurately calibrate the lesion tissue, two types of images are required for fusion processing. One is a white light image for observing a human tissue, and the other is a fluorescence image for marking a lesion. A user is more convenient to observe the human tissue through the white light image, and however the fluorescence image can only identify the human lesion tissue, so as to display the position and the boundary of the lesion. The two images are accurately fused, so that the user is convenient to observe the position and boundary position of the lesion in the human tissue.

Due to different wave lengths of the white light and the fluorescence, the fluorescence endoscope system has a two-circuit image sensor; and one-circuit image sensor is configured to collect a white light image, and the other circuit of image sensor is configured to collect a fluorescence image. The different installation positions and angles of the two-circuit different image sensor will cause that the pixel of the collected white light image cannot be aligned with that of the fluorescence image at a horizontal position and a vertical position, so that the white light image and the fluorescence image have difference in space.

At the same time, the performance parameter of the white light image sensor is quite different from that of the fluorescence image sensor, therefore the image quality collected by the two sensors is necessarily different. Different transmission paths passing through a signal and different image processing units passing through the endoscope system may cause different transmission delays of the white light image and the fluorescence image, thereby resulting in differences between the white light image and the fluorescence image in time.

### SUMMARY

The present disclosure provides an image synchronizing method, a system and a piece of electronic equipment, which can be configured to reduce differences between a white light image and a fluorescence image in a fluorescence imaging system in time and space.

According to a first aspect, the present disclosure provides an image synchronizing method, including the following steps of:
dividing N memory chips in a memory, and storing white light image data in the N memory chips circularly, wherein N is an integer greater than or equal to 2;
invoking a pre-stored frame synchronization parameter M when receiving fluorescence image data, wherein the frame synchronization parameter M is configured to adjust time delay between a fluorescence image and a white light image, and M is a positive integer less than or equal to N;
invoking the white light image data corresponding to a (X−M)^{th} memory chip from the N memory chips based on the frame synchronization parameter M, and X represents a tagged value of a memory chip; and
fusing the white light image data with the fluorescence image data, so as to obtain a first fusion image.

Under a case that the white light image and the fluorescence image generate difference, this method may compensate for the difference between the white light image and the fluorescence image in a manner that the white light image data after the difference is invoked through the frame synchronization parameter, thereby ensuring an imaging effect of a fluorescence imaging system.

In a possible design, the operation of invoking the white light image data corresponding to the (X−M)^{th} memory chip from the N memory chips based on the frame synchronization parameter M includes the following steps of:
determining a difference value X−M between the frame synchronization parameter M and the tagged value X of the memory chip, wherein M is an integer less than or equal to N; and
invoking the white light image data with the same tagged value X of the memory chip as the difference value X−M in the memory chip from the N memory chips, wherein the white light image data is the front M frame image of the currently received white light image.

In a possible design, after the operation of fusing the white light image data with the fluorescence image data, so as to obtain the first fusion image, the method further includes the following steps of:
invoking a vertical adjustment parameter, through which the first fusion image is subjected to vertical pixel adjustment; and
deleting J line pixel from a first edge of the fluorescence image in the first fusion image according to the vertical adjustment parameter, and adding J line pixel to a second edge of the fluorescence image in the first fusion image, so as to obtain a second fusion image, wherein the first edge is parallel to the second edge, and J is an integer greater than 0.

The vertical pixel adjustment for an input image in the fluorescence image system is implemented through the above method, thereby avoiding the situation that a vertical shift exists in the input image.

In a possible design, after the operation of fusing the white light image data with the fluorescence image data, so as to obtain the first fusion image, the method further includes the following steps of:
invoking a horizontal adjustment parameter, through which the first fusion image is subjected to horizontal pixel adjustment; and
deleting K row pixel from a third edge of the fluorescence image in the first fusion image according to the horizontal adjustment parameter, and adding K row pixel to a fourth edge of the fluorescence image in the first fusion image, so as to obtain a third fusion image, wherein the third edge is parallel to the fourth edge, and K is an integer greater than 0.

The horizontal pixel adjustment for the input image in the fluorescence image system is implemented through the above method, thereby avoiding the situation that a horizontal shift exists in the input image.

According to a second aspect, the present disclosure provides an image synchronizing system, which includes:
a dividing module, which is configured to divide N memory chips in a memory, and to store white light image data in the N memory chips circularly, wherein N is an integer greater than or equal to 2;
a receiving module, which is configured to invoke a pre-stored frame synchronization parameter M when receiving fluorescence image data, wherein the frame synchronization parameter is configured to adjust time delay between a fluorescence image and a white light image, and M is a positive integer less than or equal to N; and
a processing module, which is configured to invoke the white light image data corresponding to a (X−M)^{th} memory chip from the N memory chips based on the frame synchronization parameter M, and to fuse the white light image data with the fluorescence image data, so as to obtain a first fusion image, and X represents a tagged value of a memory chip.

In a possible design, the processing module is specifically configured to determine a difference value X−M between the frame synchronization parameter M and the tagged value X of the memory chip; and the white light image data with the tagged value X of the memory chip same as the difference value X−M in the memory chip is invoked from the N memory chips.

In a possible design, the processing module is also specifically configured to invoke a vertical adjustment parameter, through which the first fusion image is subjected to vertical pixel adjustment; to delete J line pixel from a first edge of the fluorescence image in the first fusion image according to the vertical adjustment parameter, and to add J line pixel to a second edge of the fluorescence image in the first fusion image, so as to obtain a second fusion image, wherein the first edge is parallel to the second edge, and J is an integer greater than 0.

In a possible design, the processing module is also specifically configured to invoke a horizontal adjustment parameter, through which the fluorescence image of first fusion image is subjected to horizontal pixel adjustment; to delete K row pixel from a third edge of the fluorescence image in the first fusion image according to the horizontal adjustment parameter, and to add K row pixel to a fourth edge of the fluorescence image in the first fusion image, so as to obtain a third fusion image, wherein the third edge is parallel to the fourth edge, and K is an integer greater than 0.

According to a third aspect, the present disclosure provides a piece of electronic equipment, which includes:
a memory, which is configured to store a computer program; and
a processor, which is configured to implement the steps of the above image synchronizing method when executing the computer program stored on the memory.

According to a fourth aspect, the present disclosure provide a computer readable storage medium, wherein the computer readable storage medium stores a computer program, and when being executed by a processor, the computer program implements the steps of the above image synchronizing method.

Various aspects in the above second aspect to the fourth aspect and the technical effects that may be achieved by various aspects refer to the specifications for the technical effects that may be achieved by the first aspect or various possible solutions in the first aspect, and repetition is not made herein.

### BRIEF DESCRIPTION OF THE DRAWINGS

FIG. 1 is a flow chart of an image synchronizing method provided by the present disclosure.

FIG. 2 is a schematic diagram of an image storage process provided by the present disclosure.

FIG. 3 is a schematic diagram of an image horizontal synchronizing effect provided by the present disclosure.

FIG. 4 is a schematic diagram of an image vertical synchronizing effect provided by the present disclosure.

FIG. 5 is a structural schematic diagram of an image processing system provided by the present disclosure

FIG. 6 is a structural schematic diagram of a piece of electronic equipment provided by the present disclosure.

### DETAILED DESCRIPTION OF THE EMBODIMENTS

In order to make the objectives, technical solution and advantages of the present disclosure clearer, the present disclosure will be described in detail below in conjunction with the drawings. The specific operation methods in the method embodiment may also be applied in a device embodiment or a system embodiment. It is noted that “a plurality of” described in the present disclosure is understood as “at least two”. “And/or” describes an association relationship of the associated objects, indicating that there may be three kinds of relationships. For example, A and/or B may indicate that A alone exists, A and B coexist, and B alone exists. Connection of A and B may indicate two situations that A is directly connected to B, and A is connected to B through C. In addition, in the description of the present disclosure, the terms “first”, “second” and the like are merely used for distinguishing the description purpose, instead of being understood as indicating or implying relative importance and indicating or implying the sequence.

In the current fluorescence imaging system, due to different wave lengths of the white light and the fluorescence, the fluorescence endoscope system has a two-circuit image sensor; and one-circuit image sensor is configured to collect a white light image, and the other circuit of image sensor is configured to collect a fluorescence image. The different installation positions and angles of the two-circuit different image sensor will cause that the pixel of the collected white light image cannot be aligned with that of the fluorescence image at a horizontal position, so that the white light image and the fluorescence image have difference necessarily in space.

At the same time, since the image quality collected by the white light image sensor is necessarily different from that collected by the fluorescence image sensor, different transmission paths passing through a signal and different image processing units passing through the endoscope system may cause different transmission delays of the white light image and the fluorescence image, thereby resulting in differences between the white light image and the fluorescence image in time.

In order to solve the difference between the white light image and the fluorescence image in time, the embodiment of the present disclosure provides an image synchronizing method, which can invoke the preset frame synchronization parameter when receiving the fluorescence image data; and the white light image data corresponding to one memory chip is invoked from N memory chips based on the frame synchronization parameter, and the white light image data is fused with the fluorescence image data, so as to obtain the first fusion image. Under a case that the white light image and the fluorescence image generate difference, this method may compensate for the difference between the white light image and the fluorescence image in a manner that the white light image data after the difference is invoked through the frame synchronization parameter, thereby ensuring an imaging effect of a fluorescence imaging system.

Various embodiments of the present disclosure are described in details below in combination with the drawings.

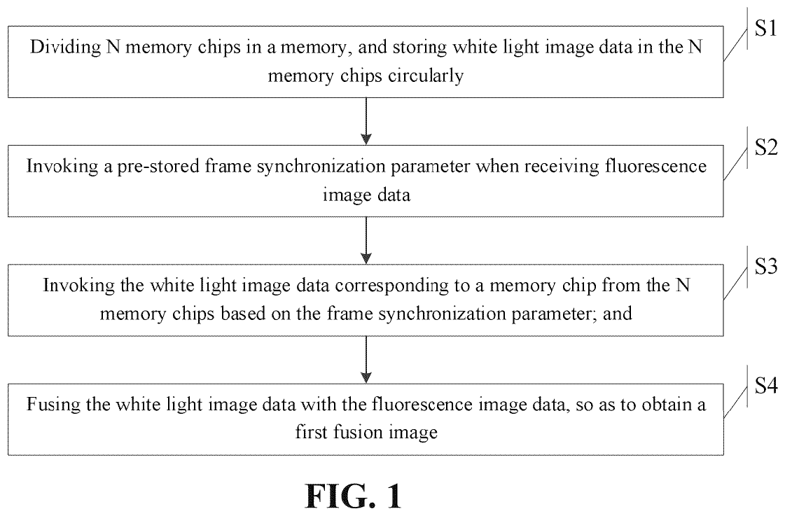

As shown in FIG. 1, which is an image synchronizing method provided by one embodiment of the present disclosure, and the specific implementing process of the method is as follows:
S1: dividing N memory chips in a memory, and storing white light image data in the N memory chips circularly;
S2: invoking a pre-stored frame synchronization parameter M when receiving fluorescence image data;
S3: invoking the white light image data corresponding to a (X−M) memory chip from the N memory chips based on the frame synchronization parameter M; and
S4: fusing the white light image data with the fluorescence image data, so as to obtain a first fusion image.

Specifically speaking, in the embodiments of the present disclosure, the frame synchronization parameter M is pre-stored in the fluorescence imaging system, and the frame synchronization parameter M may be determined through the following method:
firstly, a target is placed at the front of the fluorescence imaging system, so that the fluorescence imaging system can collect the fluorescence image and the white light image, and then the target is shaken quickly so as to collect the fused image; and in the fused image, a streaking situation generated by the fluorescence image at the target in relative to the white light image may be determined. And then the frame synchronization parameter is increased or reduced according to the streaking situation, so that the fluorescence image will not generate the streaking situation at the target in relative to the white light image.

After obtaining the frame synchronization parameter, the frame synchronization parameter is stored in a register of the fluorescence imaging system, so that the frame synchronization parameter may be directly invoked from the register when the fluorescence imaging system needs to use the frame synchronization parameter.

After the frame synchronization parameter is saved in the register based on the above method, the fluorescence image system will directly invoke the pre-stored frame synchronization parameter from the register during use when receiving the fluorescence image data.

Firstly, in the embodiments of the present disclosure, in order to improve the frame synchronization effect of the fluorescence image and the white light image, the white light image data is required to be stored in a setting memory chip. Specifically speaking, a memory is determined and divided into N memory chips, and each memory chip is configured to store the white light image data corresponding to a first frame white light image. When receiving the first frame white light image, the white light image data corresponding to the first frame white light image is saved in a first memory chip, and then the white light image data corresponding to a second frame white light image is stored in a second memory chip, in this way, various white light image data is stored in the N memory chips. When the white light image data has been stored in the N^{th} memory chip, the follow-up white light image data will start polling storage continuously from the first memory chip.

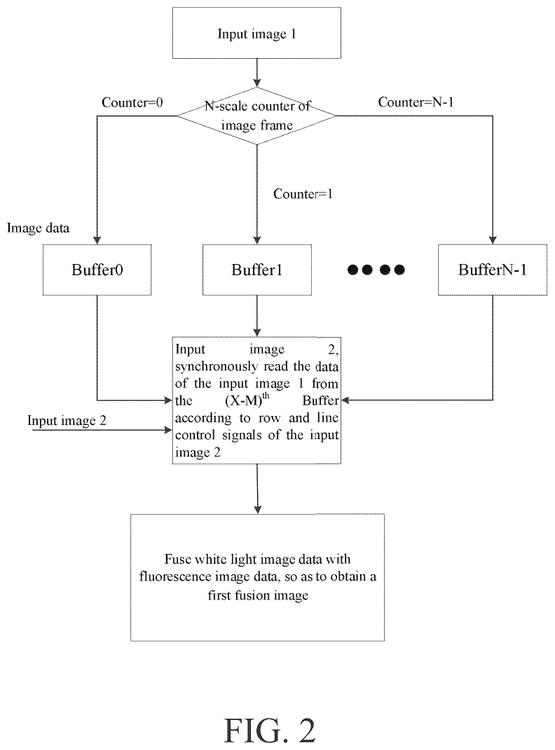

For example, the memory is divided into N storage spaces named as Buffer. As shown in FIG. 2, a frame synchronization control module controls a counter of a N-scale through a frame synchronization signal of an input white light image 1, and the counting scope of the counter is from 0 to N−1. When the counter is equal to 0, the whole image data of the white light image 1 is stored inside Buffer 0; and when the counter is equal to 1, the whole image data of the white light image 1 is stored inside Buffer 1, and so on. When the counter is equal to N−1, the whole image data of the white light image 1 is stored in Buffer N−1.

Based on the above storage method for the white light image data, when inputting the fluorescence image 2, the difference value between the frame synchronization parameter M and the tagged value X of the memory chip where the current input white light image is located is determined, and the white light image data with the tagged value X of the memory chip same as the difference value X−M in the (X−M)^{th} memory chip is invoked from the N memory chips.

Referring to the method as shown in FIG. 2, when the frame synchronization parameter is M, the difference value is determined as X−M, and at this time, the white light image data is read from Buffer X−M.

After the white light image data is invoked from the memory chip, the white light image data is fused with the fluorescence image data, so as to obtain the first fusion image, and this fusion process ensures that the pixel of the white light image is aligned with that of the fluorescence image. It is noted herein that the white light image data is selected according to the frame synchronization parameter when determining the white light image data, therefore the time delay problem between the white light image and the fluorescence image is avoided.

The frame synchronization of the white light image and the fluorescence image in the fluorescence imaging system may be implemented through the above method, thereby avoiding the streaking phenomenon between the fluorescence image and the white light image.

Further, in an alternative solution, after the first fusion image is obtained through frame synchronization processing, the first fusion image may be subjected to the horizontal pixel adjustment.

Specifically speaking, the horizontal adjustment parameter, through which the first fusion image is subjected to horizontal pixel adjustment is invoked; the K row pixel is deleted from the third edge of the fluorescence image in the first fusion image, and the K row pixel is added to the fourth edge of the fluorescence image in the first fusion image, so as to obtain the second fusion image. The first edge herein is parallel to the second edge.

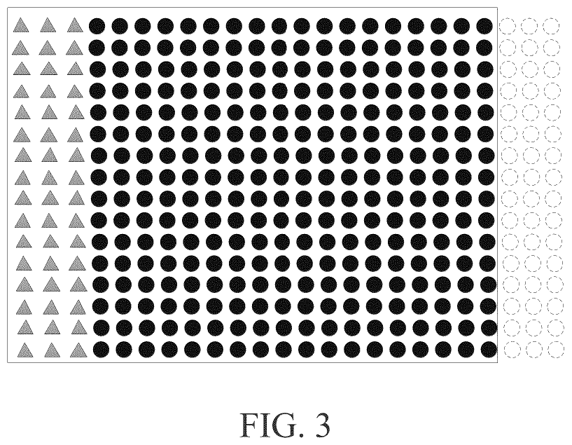

For example, as shown in FIG. 3, after inputting the image in the fluorescence imaging system, judge whether the image needs to move in a horizontal direction. If so, move K pixel points to the right, delete K pixel points on the rightmost side of each line, fill K pixels with 0 value on the leftmost side of each line, and output the second fusion image with adjusted pixel. K is an integer greater than 0.

If needing to move K pixel points to the left, delete K pixel points on the leftmost side of each line, fill K pixels with 0 value on the rightmost side of each line, and output the second fusion image with adjusted pixel.

The horizontal pixel adjustment of the input image in the fluorescence image system is implemented through the above method, thereby avoiding the situation that a horizontal shift exists in the input image.

Further, in an alternative embodiment, in addition to performing horizontal pixel adjustment on the input image, the first fuse image may be subjected to vertical pixel adjustment.

Specifically speaking, the vertical adjustment parameter, through which the first fusion image is subjected to vertical pixel adjustment is invoked; the J row pixel is deleted from the first edge of the fluorescence image in the first fusion image, and the J row pixel is added to the fourth edge of the fluorescence image in the first fusion image, so as to obtain the second fusion image. The third edge herein is parallel to the fourth edge, and J is an integer greater than 0.

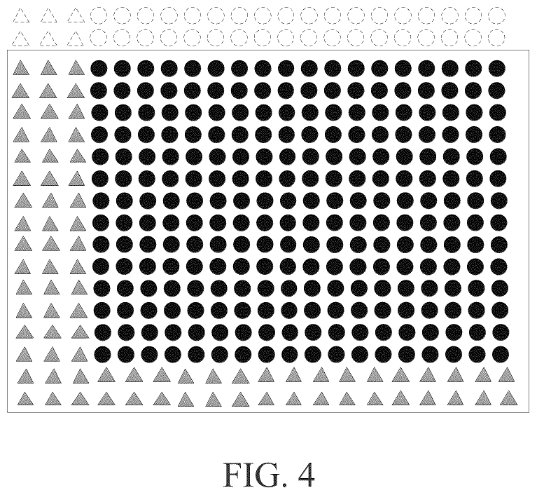

For example, as shown in FIG. 4, after inputting the image in the fluorescence imaging system, judge whether the image needs to move in a vertical direction. If so, move J pixel points upwards, delete J pixel points at the uppermost of each line, fill J pixels with 0 value on the lowermost side of each line, and output the second fusion image with adjusted pixel.

If needing to move J pixel points downwards, delete J pixel points at the lowermost of each line, fill J pixels with 0 value at the uppermost side of each row, and output the second fusion image with adjusted pixel.

The vertical pixel adjustment for the input image in the fluorescence image system is implemented through the above method, thereby avoiding the situation that a vertical shift exists in the input image.

It is noted that, in the embodiments of the present disclosure, the adjustment sequence for the frame synchronization adjustment, the horizontal parameter adjustment and the vertical parameter adjustment of the input image may be adjusted, for example, after the horizontal parameter adjustment and the vertical parameter adjustment are performed firstly, the frame synchronization adjustment is performed. Therefore, the adjustment sequence of these three adjustment methods is not limited in the embodiments of the present disclosure, and the adjustment may be made according to the needs in the specific application scenarios.

In conclusion, in the embodiments of the present disclosure, a preset frame synchronization parameter can be invoked when receiving fluorescence image data; and the white light image data corresponding to one memory chip is invoked from N memory chips based on the frame synchronization parameter, and the white light image data is fused with the fluorescence image data, so as to obtain the first fusion image. Under a case that the white light image and the fluorescence image generate difference, this method may compensate for the difference between the white light image and the fluorescence image in a manner that the white light image data after the difference is invoked through the frame synchronization parameter, thereby ensuring the imaging effect of the fluorescence imaging system.

In addition, the input image may also be subjected to horizontal pixel adjustment and vertical pixel adjustment, so as to ensure that the input image does not generate a horizontal offset and a vertical offset, and to improve the synchronization effect of the input image.

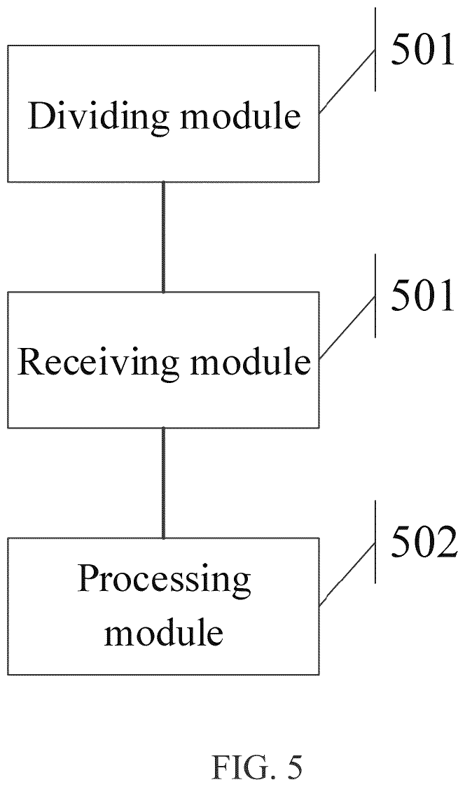

Based on the same inventive concept, the present disclosure provides an image synchronizing system, and referring to FIG. 5, the system includes:
a dividing module 501, which is configured to divide N memory chips in a memory, and to store white light image data in the N memory chips circularly, wherein N is an integer greater than or equal to 2;
a receiving module 502, which is configured to invoke a pre-stored frame synchronization parameter M when receiving fluorescence image data, wherein the frame synchronization parameter is configured to adjust time delay between a fluorescence image and a white light image, and M is a positive integer less than or equal to N; and
a processing module 503, which is configured to invoke the white light image data corresponding to a (X−M)^{th} memory chip from the N memory chips based on the frame synchronization parameter M, and to fuse the white light image data with the fluorescence image data, so as to obtain a first fusion image, and X represents a tagged value of a memory chip.

In an alternative embodiment, the processing module 503 is specifically configured to determine a difference value X−M between the frame synchronization parameter M and the tagged value X of the memory chip where the current input white light image is located, the white light image data with the tagged value X of the memory chip same as the difference value X−M in the memory chip is invoked from the N memory chips, and the white light image data is the front M frame image of the currently received white light image.

In an alternative embodiment, the processing module 503 is also specifically configured to invoke a vertical adjustment parameter, through which the first fusion image is subjected to vertical pixel adjustment; to delete J line pixel from a first edge of the fluorescence image in the first fusion image according to the vertical adjustment parameter, and to add J line pixel to a second edge of the fluorescence image in the first fusion image, so as to obtain a second fusion image, wherein the first edge is parallel to the second edge, and J is an integer greater than 0.

In an alternative embodiment, the processing module 503 is also specifically configured to invoke a horizontal adjustment parameter, through which the first fusion image is subjected to horizontal pixel adjustment; to delete K line pixel a third edge of the fluorescence image in the first fusion image according to the horizontal adjustment parameter, and to add K line pixel to a fourth edge of the fluorescence image in the first fusion image, so as to obtain a third fusion image, wherein the third edge is parallel to the fourth edge, and K is an integer greater than 0.

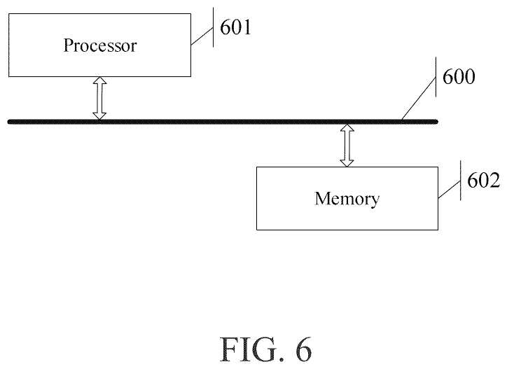

Based on the same inventive concept, the present disclosure further provides a piece of electronic equipment, which may implement the functions of the above image synchronizing system, and referring to FIG. 6, the electronic equipment includes:
at least one processor 601, and a memory 602 connected to at least one processor 601; the specific connecting medium between the processor 601 and the memory 602 is not limited in the embodiments of the present disclosure, and FIG. 6 is an example that the processor 601 is connected to the memory 602 through a bus 600. The bus 600 is shown in FIG. 6 as a thick line, and the connecting mode for other components is merely illustrative, instead of a limitation. The bus 600 may be divided into an address bus, a data bus, a control bus and the like. For easy expression, the bus 600 is merely indicated by a thick line in FIG. 6, but not indicating that only one bus or one bus type is provided. Or, the processor 601 may also be called a controller, and the name is not limited.

In the embodiments of the present disclosure, the memory 602 stores an instruction that may be executed by at least one processor 601, and at least one processor 601 may execute the image synchronizing method described above by executing the instruction stored in the memory 602. The processor 601 may implement the functions of various modules in the system shown in FIG. 5.

Wherein the processor 601 is a control center of the device, and connects all parts of the entire control equipment by using various interfaces and lines. By running or executing the instruction stored in the memory 602 and invoking data stored in the memory 602, the processor 601 performs various functions of the device and data processing, thereby performing overall monitoring on the device.

In a possible design, the processor 601 may include one or more processing units. The processor 601 may be integrated with an application processor and a modem processor. The application processor mainly processes an operating system, a user interface, an application program, and the like, and the modem processor mainly processes wireless communication. It can be understood that the foregoing modem processor may not be integrated into the processor 601. In some embodiments, the processor 601 and the memory 602 may be implemented on the same chip, and in some embodiments, the processor 601 and the memory 602 may also be implemented in respective on the separate chips.

The processor 601 may be a general-purpose processor, such as a central processing unit (CPU), a digital signal processor, an application-specific integrated circuit, a field-programmable gate array or other programmable logic devices, a discrete gate or a transistor logic device and a discrete hardware module, which may implement or execute various methods, steps and logic diagrams disclosed in the embodiments of the present disclosure. The general-purpose processor may be a microprocessor or any conventional processor, etc. The steps of the image synchronizing method disclosed in combination with the embodiments of the present disclosure may be directly embodied to be executed and completed by a hardware processor or executed and completed by a combination of hardware and software modules in the processor.

As a non-volatile computer readable storage medium, the memory 602 may be used to store a non-volatile software program, a non-volatile computer executable program and a module. The memory 602 may include at least one type of storage medium, such as may include a flash memory, a hard disk, a multimedia card, a card memory, a random access memory (RAM), a static random access memory (SRAM), a programmable read only memory (PROM), a read only memory (ROM), an electrically erasable programmable read-only memory (EEPROM), a magnetic memory, a disk, an optical disk, etc. The memory 602 is a program code capable of carrying or storing an expectation with an instruction or data structure fonn and any other medium accessed by a computer, but it is not limited to this. The memory 602 in the embodiments of the present disclosure may also be a circuit or any other devices capable of implementing storage functions, so as to store a program instruction and/or data.

A code corresponding to an image synchronizing method introduced in the foregoing embodiments may be solidified to a chip through designing and programming the processor 601, so that the chip can execute the steps of the image synchronizing method of the embodiment shown in FIG. 1 when running. How to design and program the processor 601 is a technology commonly known by those skilled in the art, so repetition is not made herein.

Based on the same inventive concept, embodiments of the present disclosure further provide a storage medium, which stores a computer instruction, and when the computer instruction runs on a computer, the computer executes the image synchronizing method described above.

In some possible implementation modes, various aspects of the image synchronizing method provided by the present disclosure may also implement a form of a program product, including a program code. When the program product runs on the device, the program code is used to enable the control equipment to execute the steps of the image synchronizing method of various exemplary implementation modes of the present disclosure described in the specification.

Those skilled in the art may understand that embodiments of the present disclosure may be provided as methods, systems, or computer program products. Therefore, the present disclosure may adopt forms of complete hardware embodiments, complete software embodiments or embodiments integrating software and hardware. Moreover, the present disclosure may adopt the form of a computer program product implemented on one or more computer available storage media (including but not limited to a disk memory, a compact disc read only memory (CD-ROM), an optical memory, etc.) containing computer available program codes.

The present disclosure is described with reference to flowcharts and/or block diagrams of the method, the device (system) and the computer program product according to the embodiments of the present disclosure. It should be understood that each flow and/or block in the flowchart and/or block diagram, and the combination of the flow and/or block in the flowchart and/or block diagram can be implemented by the computer program instructions. These computer program instructions can be provided to a processor of a general-purpose computer, a special-purpose computer, an embedded processor or other programmable data processing devices to generate a machine, so that instructions which are executed by the processor of the computer or other programmable data processing devices generate a device which is used for implementing the specified functions in one or more flows of the flowchart and/or one or more blocks of the block diagram.

These computer program instructions may also be stored in the computer readable memory which can guide the computer or other programmable data processing devices to work in a particular way, so that the instructions stored in the computer-readable memory generate a product including an instruction device. The instruction device implements the specified functions in one or more flows of the flowchart and/or one or more blocks of the block diagram.

These computer program instructions may also be loaded on the computer or other programmable data processing devices, so that a series of operation steps are performed on the computer or other programmable data processing devices to generate the processing implemented by the computer, and the instructions executed on the computer or other programmable data processing devices provide the steps for implementing the specified functions in one or more flows of the flowchart and/or one or more blocks of the block diagram.

It is apparent that those skilled in the art may make any modification and variation to the present disclosure without deviating from the spirit and scope of the present disclosure. Thus, if these modifications and variations of the present disclosure belong to the scope of the claims and equivalent technology thereof of the present disclosure, the present disclosure is intended to include these modifications and variations.

What is claimed is:

1. An image synchronizing method, wherein the method comprises the following steps of:
dividing N memory chips in a memory, and storing white light image data in the N memory chips circularly, wherein N is an integer greater than or equal to 2;
invoking a pre-stored frame synchronization parameter M when receiving fluorescence image data, wherein the frame synchronization parameter M is configured to adjust time delay between a fluorescence image and a white light image, and M is a positive integer less than or equal to N;
invoking the white light image data corresponding to a (X−M)^{th} memory chip from the N memory chips based on the frame synchronization parameter M, and X represents a tagged value of a memory chip; and
fusing the white light image data with the fluorescence image data, so as to obtain a first fusion image.

2. The method according to claim 1, wherein the operation of invoking the white light image data corresponding to the (X−M)^{th} memory chip from the N memory chips based on the frame synchronization parameter M comprises the following steps of:
determining a difference value X−M between the frame synchronization parameter M and a tagged value X of the memory chip, wherein M is an integer less than or equal to X; and
invoking the white light image data with the same tagged value X of the memory chip as the difference value X−M in the memory chip from the N memory chips, wherein the white light image data is the front M frame image of the currently received white light image.

3. The method according to claim 1, wherein after the operation of fusing the white light image data with the fluorescence image data, so as to obtain the first fusion image, the method further comprises the following steps of:
invoking a vertical adjustment parameter, through which the first fusion image is subjected to vertical pixel adjustment; and
deleting J line pixel from a first edge of the fluorescence image in the first fusion image according to the vertical adjustment parameter, and adding J line pixel to a second edge of the fluorescence image in the first fusion image, so as to obtain a second fusion image, wherein the first edge is parallel to the second edge, and J is an integer greater than 0.

4. The method according to claim to 2, wherein after the operation of fusing the white light image data with the fluorescence image data, so as to obtain the first fusion image, the method further comprises the following steps of:
invoking a vertical adjustment parameter, through which the first fusion image is subjected to vertical pixel adjustment; and
deleting J line pixel from a first edge of the fluorescence image in the first fusion image according to the vertical adjustment parameter, and adding J line pixel to a second edge of the fluorescence image in the first fusion image, so as to obtain a second fusion image, wherein the first edge is parallel to the second edge, and J is an integer greater than 0.

5. The method according to claim 1, wherein after the operation of fusing the white light image data with the fluorescence image data, so as to obtain the first fusion image, the method further comprises the following steps of:
invoking a horizontal adjustment parameter, through which the first fusion image is subjected to horizontal pixel adjustment; and
deleting K row pixel from a third edge of the fluorescence image in the first fusion image according to the horizontal adjustment parameter, and adding the K row pixel to a fourth edge of the fluorescence image in the first fusion image, so as to obtain a third fusion image, wherein the third edge is parallel to the fourth edge, and K is an integer greater than 0.

6. The method according to claim 2, wherein after the operation of fusing the white light image data with the fluorescence image data, so as to obtain the first fusion image, the method further comprises the following steps of:
invoking a horizontal adjustment parameter, through which the first fusion image is subjected to horizontal pixel adjustment; and
deleting K row pixel from a third edge of the fluorescence image in the first fusion image according to the horizontal adjustment parameter, and adding the K row pixel to a fourth edge of the fluorescence image in the first fusion image, so as to obtain a third fusion image, wherein the third edge is parallel to the fourth edge, and K is an integer greater than 0.

7. An image synchronizing system, wherein the system comprises:
a dividing module, which is configured to divide N memory chips in a memory, and to store white light image data in the N memory chips circularly, wherein N is an integer greater than or equal to 2;
a receiving module, which is configured to invoke a pre-stored frame synchronization parameter M when receiving fluorescence image data, wherein the frame synchronization parameter is configured to adjust time delay between a fluorescence image and a white light image, and M is a positive integer less than or equal to N; and
a processing module, which is configured to invoke the white light image data corresponding to a (X−M)^{th} memory chip from the N memory chips based on the frame synchronization parameter M, and to fuse the white light image data with the fluorescence image data, so as to obtain a first fusion image, and X represents a tagged value of the memory chip.

8. The system according to claim 5, wherein the processing module is specifically configured to determine a difference value X−M between the frame synchronization parameter M and the tagged value X of the memory chip, and M is an integer less than or equal to X; and the white light image data with the tagged value X of the memory chip same as the difference value X−M in the memory chip is invoked from the N memory chips, and the white light image data is the front M frame image of the currently received white light image.

9. The system according to claim 5, wherein the processing module is also specifically configured to invoke a vertical adjustment parameter, through which the first fusion image is subjected to vertical pixel adjustment; to delete J line pixel from a first edge of the fluorescence image in the first fusion image according to the vertical adjustment parameter, and to add J line pixel to a second edge of the fluorescence image in the first fusion image, so as to obtain a second fusion image, and the first edge is parallel to the second edge, and J is an integer greater than 0.

10. The system according to claim to 6, wherein the processing module is also specifically configured to invoke a vertical adjustment parameter, through which the first fusion image is subjected to vertical pixel adjustment; to delete J line pixel from a first edge of the fluorescence image in the first fusion image according to the vertical adjustment parameter, and to add J line pixel to a second edge of the fluorescence image in the first fusion image, so as to obtain a second fusion image, and the first edge is parallel to the second edge, and J is an integer greater than 0.

11. The system according to claim 5, wherein the processing module is also specifically configured to invoke a horizontal adjustment parameter, through which the first fusion image is subjected horizontal pixel adjustment, to delete K row pixel from a third edge of the fluorescence image of the first fusion image according to the horizontal adjustment parameter, and to add the K row pixel to a fourth edge of the fluorescence image of the first fusion image, so as to obtain a third fusion image, and the third edge is parallel to the fourth edge, and K is an integer greater than 0.

12. The system according to claim 6, wherein the processing module is also specifically configured to invoke a horizontal adjustment parameter, through which the first fusion image is subjected horizontal pixel adjustment, to delete K row pixel from a third edge of the fluorescence image of the first fusion image according to the horizontal adjustment parameter, and to add the K row pixel to a fourth edge of the fluorescence image of the first fusion image, so as to obtain a third fusion image, and the third edge is parallel to the fourth edge, and K is an integer greater than 0.

13. A piece of electronic equipment, comprising:
a memory, which is configured to store a computer program; and
a processor, which is configured to implement the steps of the above method in claim 1 when executing the computer program stored on the memory.

14. Apiece of electronic equipment, comprising:
a memory, which is configured to store a computer program; and
a processor, which is configured to implement the steps of the above method in claim 2 when executing the computer program stored on the memory.

15. Apiece of electronic equipment, comprising:
a memory, which is configured to store a computer program; and
a processor, which is configured to implement the steps of the above method in claim 3 when executing the computer program stored on the memory.

16. Apiece of electronic equipment, comprising:
a memory, which is configured to store a computer program; and
a processor, which is configured to implement the steps of the above method in claim 4 when executing the computer program stored on the memory.

17. A computer readable storage medium, wherein the computer readable storage medium stores a computer program, and when executed by a processor, the computer program implements the steps of the method in claim 1.

18. A computer readable storage medium, wherein the computer readable storage medium stores a computer program, and when executed by a processor, the computer program implements the steps of the method in claim 2.

19. A computer readable storage medium, wherein the computer readable storage medium stores a computer program, and when executed by a processor, the computer program implements the steps of the method in claim 3.

20. A computer readable storage medium, wherein the computer readable storage medium stores a computer program, and when executed by a processor, the computer program implements the steps of the method in claim 4.
